(19) United States
(12) Patent Application Publication
(10) Pub. No.: US 2022/0313134 A1
(43) Pub. Date: Oct. 6, 2022

(54) BIOSENSING GARMENT AND METHOD

(71) Applicant: Prevayl Innovations Limited, Manchester (GB)

(72) Inventor: Isabel Rose Hepworth, Manchester (GB)

(21) Appl. No.: 17/615,904

(22) PCT Filed: Jul. 10, 2020
(86) PCT No.: PCT/GB2020/051662

§ 371 Date: Dec. 2, 2021

(30) Foreign Application Priority Data
Jul. 12, 2019 (GB) 1910048.6
Aug. 15, 2019 (GB) 1911722.5

(51) Int. Cl. A61B 5/282; A41D 1/00; A41D 27/20; A61B 5/00

(52) U.S. Cl. CPC A61B 5/282; A41D 1/005; A41D 27/205; A61B 5/6804; A61B 5/0022; A61B 2562/12

(57) ABSTRACT

A biosensing garment (100). The biosensing garment (100) comprises a garment (101). The biosensing garment (100) comprises an inner biosensing textile (200) disposed within the garment (101). The inner biosensing textile (200) comprises a textile panel (201). The inner biosensing textile comprises a biosensing unit (215) positioned on the textile panel (201) for measuring a biosignal of the wearer. A first region of the textile panel (201) is attached to the garment (101) such that the first region is unable to move relative to the garment (101). A second region of the textile panel (201) is able to move relative to the garment (101).

### CROSS-REFERENCE TO RELATED APPLICATIONS

This application claims priority from United Kingdom Patent Application number 1910048.6 filed on 12 Jul. 2019 and United Kingdom Patent Application number 1911722.5 filed on 15 Aug. 2019, the whole contents of which are incorporated herein by reference.

### BACKGROUND

The present invention is directed towards a biosensing garment and method of making the same. The present invention is directed in particular towards providing a biosensing garment with an inner biosensing textile.

Garments incorporating sensors are wearable electronics designed to interface with a wearer of the garment, and to determine information such as the wearer's heart rate, rate of respiration, activity level, and body positioning. Such properties can be measured with a sensor assembly that includes a sensor for signal transduction and/or microprocessors for analysis. Such garments are commonly referred to as ‘biosensing garments’ or ‘smart clothing’. A drawback of many biosensing garments is that they are uncomfortable to the wearer and have an unattractive outward appearance due to the presence of the electronic components.

It is desirable to overcome at least some of the problems associated with the prior art, whether explicitly discussed herein or otherwise.

### SUMMARY

According to the present disclosure there is provided a biosensing garment and method as set forth in the appended claims. Other features of the invention will be apparent from the dependent claims, and the description which follows.

According to a first aspect of the disclosure, there is provided a biosensing garment. The biosensing garment comprises a garment. The biosensing garment comprises an inner biosensing textile disposed within the garment. The inner biosensing textile comprises a textile panel. The inner biosensing textile comprises a biosensing unit positioned on the textile panel for measuring a biosignal of the wearer. A first region of the textile panel is attached to the garment such that the first region is unable to move relative to the garment. A second region of the textile panel is able to move relative to the garment.

Here, “biosignal” may refer to any signal in a living being that can be measured and monitored. The term “biosignal” is not limited to electrical signals and can refer to other forms of non-electrical biosignals. A biosensing unit therefore refers to an electronic component that is able to measure a biosignal of the wearer. The biosensing unit may comprise one or more electrodes but is not limited to this arrangement.

Beneficially, the present disclosure provides a biosensing garment with an inner biosensing textile. The inner biosensing textile comprises a biosensing unit for performing biosensing. The biosensing unit is not visible from the outside of the garment and does not affect the outward appearance of the garment. Significantly still, a first region of the textile panel is attached to the garment while another region of the textile panel is able to move relative to the garment. Beneficially, this means that a part of the textile panel is attached to the garment while another part is free to move relative to the garment. The inner biosensing textile therefore does not pull on the garment when the wearer moves as part of the textile panel is able to move relative to the garment. As such, the inner biosensing textile is able to perform the required biosensing measurements with minimal effect on the comfort of the wearer even during wearer motion. Further, as the inner biosensing textile does not or does not significantly pull on the garment, the inner biosensing textile has a minimal effect on the outward appearance of the garment. Therefore, the biosensing garment is more comfortable both visually and physically to wear.

The inner biosensing textile may comprise a holder arranged to releasably hold an electronic component.

The holder/biosensing textile may comprise a first textile layer and a second textile layer. The second textile layer may be attached to a first surface of the first textile layer to define an internal cavity. The internal cavity may be for receiving the electronic component. The electronic component may be disposed within the cavity.

The biosensing textile may comprise a first textile layer and a second textile layer. The second textile layer may be attached to the first textile layer to define an internal cavity. The internal cavity may be for receiving the electronic component. The electronic component may be disposed within the cavity. The first textile layer and second textile layer may form the holder. The holder may comprise the first textile layer and the second textile layer.

The first textile layer may comprise a first surface. The second textile layer may be attached to the first surface of the first textile layer to define the internal cavity. The first surface may face in to the internal cavity. The first textile layer may comprise a second surface that faces away from the internal cavity.

The biosensing unit may be positioned on a second surface of the first textile layer opposite to the first surface of the first textile layer such that the biosensing unit is positioned outside of the internal cavity. The biosensing unit may be conductively connected to the electronic component through the first textile layer. Beneficially, the first and second textile layers define an internal cavity in which the electronic component is disposed. The internal cavity protects the electronic component. The biosensing unit is not disposed within the internal cavity but is instead positioned or incorporated into the first surface of the first textile layer. This enables the biosensing unit to be brought towards or into contact with the skin of a wearer of the biosensing textile so as to enable the biosignal to be measured.

The electronic component may comprise a power source. The electronic component may comprise a controller for controlling the biosensing unit. The electronic component may comprise a communicator for communicating with an external device. The electronic component may comprise a combination of two or more of the power source, controller, and communicator.

The internal cavity may be in the form of a pocket with an opening such that at least a part of the electronic component may be accessed and/or removed. This may be beneficial if the electronic component needs to be accessed to, for example, charge a power source. The first and second textile layers may comprise at least one fastener such as a zip or clasp to enable the opening to be selectively closed. The fastener may be a waterproof fastener such as a waterproof zip. This means that when the opening is closed, the waterproof fastener protects against water ingress into the opening. Beneficially, this helps protect the electronic components against water ingress such as when the biosensing textile is washed. The internal cavity may be sealed. For example, the edges of the second textile layer may be joined to the first textile layer such that there is no opening between the first and second textile layer. The second textile layer may be adhered or welded to the first textile layer. Welding the first textile layer to the second textile layer may comprise applying heat and/or pressure to the first textile layer and/or the second textile layer so as to cause the edges of the first textile layer to be attached to the second textile layer.

The textile panel may comprise means to position the biosensing unit away from the garment such that, when worn, the biosensing unit is positioned on or near the body surface.

The means may comprise the textile panel being shaped to position the biosensing unit away from the garment such that, when worn, the biosensing unit is positioned on or near the body surface. This means that the textile panel has a three-dimensional shape. Beneficially, the textile panel is shaped to urge the biosensing unit towards the body surface when worn. This helps maintain the biosensing unit in close proximity to the body surface and, in some applications, in contact with the body surface.

The textile panel may comprise a dart. The dart may act to shape the textile panel such that the biosensing unit is positioned away from the garment. This means that the dart enables the textile panel to be shaped such that, when worn, the biosensing unit is positioned on or near the body surface.

The textile panel may comprise a seam. The seam may act to shape the textile such that the biosensing unit is positioned away from the garment. This means that the seam enables the textile panel to be shaped such that, when worn, the biosensing unit is positioned on or near the body surface.

The means may comprise an electrostatic generator for generating static electricity so as to cause the textile panel to cling to the wearer.

The means may comprise one or more weights for urging the textile panel down and towards the wearer. That is, the textile panel may be weighted. The textile panel may be weighted by use of one or more weighted elements (such as metal weights) that are attached to the textile panel. The weighted elements may be stitched into the textile panel or adhered to the textile panel.

The textile panel may comprise one or more gripper sections provided on the underside surface of the biosensing textile that grip the biosensing textile to the skin surface, e.g. by friction. In one example, the gripper sections may be provided by silicone material such as silicone tape. The gripper section may provide the means to position the biosensing unit away from the garment.

The first portion of the textile panel may be an end region of the textile panel. The second portion of the textile panel may be a remaining portion of the textile panel. That is, the remaining portion of the textile panel may be able to move relative to the garment. This may mean that only the end portion of the textile panel is unable to move relative to the garment.

The biosensing unit may be attached to or may be integral with the textile panel. The biosensing unit may be disposed on an inner surface of the textile panel such that the biosensing unit is able to be positioned in contact with the body surface when the garment is worn. This is not required when, for example, the biosensing unit does not require skin contact to measure a biosignal.

The garment may be a top. The first region of the textile panel may be attached to the top at a position corresponding to a shoulder of the wearer such that, when worn, the first region of the textile panel is proximate to the shoulder of the wearer. The textile panel and/or the inner biosensing textile may only be attached to the garment via the first region and as such may only be attached to the shoulder region of the garment. Generally, the shoulder region is subject to minimal movement and therefore is a stable point to attach the textile panel to garment without affect the visual appearance of the outer garment.

The textile panel may be attached to the garment using a twin needle top stitch. The twin needle top stitch may be provided on either side of a seam of the garment. The textile panel may be attached to a shoulder region of the garment (e.g. when the garment is a top) via a twin needle top stitch provided on either side of a seam of the garment at the shoulder region. The other should region of the garment may also be provided with a twin needle top stitch provided on either side of a seam of the garment so as to balance out the visual appearance of the garment.

The biosensing unit may be positioned on the textile panel at a location corresponding to a left chest region of the wearer such that, when worn, the biosensing unit is positioned proximate to the cardiac region of the wearer.

The textile panel may comprise a plurality of biosensing units. The biosensing unit may be a first biosensing unit. The biosensing textile may further comprise a second biosensing unit positioned on the textile panel for measuring a biosignal of the wearer. The first and second biosensing units may cooperate to measure a biosignal of the wearer.

The second biosensing unit may be located at a position corresponding to a central chest region of the wearer such that, when worn, the second biosensing unit is proximate to the central chest region of the wearer.

The inner biosensing textile may further comprises a second textile panel; and a biosensing unit positioned on the second textile panel. The second textile panel may be joined to the first textile panel. A first region of the second textile panel may be attached to the garment such that the first region is unable to move relative to the garment. A second region of the second textile panel may be able to move relative to the garment. The first region may be an end region of the second textile panel. The second region may be a remaining portion of the second textile panel. That is, only the end region of the second textile panel may be attached to the garment and the remaining portion of the second textile panel may be able to move relative to the garment. The first textile panel may be a front textile arranged to be positioned close to the front (e.g. the chest) of the wearer. The second textile panel may be a rear textile arranged to be positioned close the rear (e.g. the back) of the wearer.

The second textile panel may be shaped to position the biosensing unit away from the garment such that, when worn, the biosensing unit is positioned on or near the body surface. The second textile panel may comprise a dart or seam.

The first textile panel and second textile panel may together form a loop or U-shaped band that is attached at both ends to a region of the garment. The region of the garment may be the shoulder region of the garment. That is, when worn, the first textile panel may be disposed on the front size of the wearer and the second textile panel may be disposed on the rear side of the wearer. This arrangement helps to divide the weight of the biosensing inner layer evenly.

The second textile panel and the first textile panel may together define an aperture through which a part of the wearers body may be received. The aperture may be an arm hole for receiving an arm of the wearer. The aperture may be a leg hole for receiving a leg of the wearer.

The textile panel may be bias cut. The first textile panel and/or the second textile panel may be bias cut. The bias cut allows the textile panel to accommodate movement without stretch fabric which would compromise the panel stability.

The garment or the biosensing textile may further comprise a power source or a plurality of power sources. The power source may be positioned on the textile panel. The power source may be for powering the biosensing unit.

The garment may further comprise an access region at a location corresponding to the location of the electronic component on the textile panel. The electronic component may be the biosensing unit or another electronic component. The access region may be arranged such that at least a part of the electronic component is visible on an outside surface of the garment. The access region may be an aperture, transparent region or translucent region on the garment.

The electronic component may comprise a power source. The electronic component may comprise a controller for controlling the biosensing unit. The electronic component may comprise both the power source and the controller. The electronic component may comprise a display. The display may be arranged to indicate a status of the biosensing garment. The display may be arranged to display information relating to the data recorded by the biosensing garment.

The electronic component may comprise an indicator arranged to indicate a status of the electronic component. The electronic component may comprise an actuator such as a button for controlling the electronic component, e.g. to turn the electronic component on/off.

The garment may comprise the aperture, and the aperture may be sized to receive the electronic component such that at least a part of the electronic component is accessible via the outside surface of the garment. The electronic component may be removable from the textile panel. The textile panel may comprise a holder for receiving the power source.

The textile panel may comprise a holder arranged to releasably hold the electronic component. The electronic component may be incorporated into or have the appearance of a fastener. The fastener may be a button, clasp, toggle, stud, snap fastener, popper, eyelet or, buckle.

The garment may be a free-form (loose-fit) garment. A free-form garment will be understood as referring to a garment that is not skin-tight and is not a compression garment.

The biosensing unit may comprise one or more electrodes.

The biosensing unit may be formed of or comprise a 2D electrically conductive material. The 2D material may be a carbon-based material. The carbon-based material may comprise graphene, e.g. pristine graphene, and/or reduced graphene oxide. The carbon-based material may be graphene and/or reduced graphene oxide in combination with one or more additional conductive agents. The carbon-based material may be a graphene derivative. In some examples. there may be additional electrically conductive agents present in the biosensing unit such as metallic components (e.g. silver precursor, silver nanoparticles, carbon nanotubes, or poly(3,4-ethylenedioxythiophene) polystyrene sulfonate (PEDOT/PSS)).

The biosensing unit may be a textile-based biosensing unit which optionally incorporates a 2D electrically conductive material into the fibre/yarn. The 2D material may be incorporated by a dyeing process in which a liquid composition containing the 2D material is contacted with the fibre/yarn. The yarn may be based on synthetic materials such as polyester, nylon, and viscose or may be based on natural materials such as cotton and wool. The yarn may be based on a combination of natural and synthetic materials. Therefore, a 2D material may be incorporated into a yarn in order to produce a yarn which is capable of conducting electricity and forming a biosensing unit.

In some examples, the yarn forming the biosensing unit may be a graphene yarn. That is, a yarn constructed entirely, essentially or substantially of graphene, e.g. graphene fibres.

The inner biosensing textile may comprise a controller in communication with the biosensing unit and operable to control the biosensing unit. The controller may control the biosensing unit to receive measurement signals. The controller may be positioned on the textile panel. The controller may be spaced apart from the biosensing unit on the textile panel. The controller may be positioned on an outer surface of the textile panel. The outer surface of the textile panel faces the garment. The controller may be positioned on the outer surface of the textile panel while the biosensing unit may be positioned on the inner surface of the textile panel. The controller may be removable from the textile panel. The textile panel may comprise a holder arranged to releasably hold the controller.

The controller may be wirelessly connected to the biosensing unit. That is, the biosensing unit may comprise a communicator for wireless communication with the controller. The controller may be conductively connected to the biosensing unit.

The controller may be conductively connected to the biosensing unit by a conductor. The conductor may be incorporated into the textile. The conductor may be an electrically conductive track or film. The controller may be provided on the garment and may not be provided on the inner biosensing textile. The power source may be conductively connected to the controller by a conductor.

The conductor may be formed of a 2D material. The conductor may be formed of the same material or in the same manner as the biosensing unit of the textile.

The conductor may be a conductive transfer. The conductive transfer may comprise a first non-conductive ink layer and a second non-conductive ink layer. An electrically conductive layer may be positioned between the first non-conductive ink layer and the second non-conductive ink layer. The conductive transfer may be adhered to the textile via use of an adhesive layer so as to form the conductor on the textile. The electrically conductive layer may comprise graphene.

The conductor may be formed from a fibre or yarn of the textile. This may mean that an electrically conductive materials such as graphene is incorporated into the fibre/yarn. In some examples, the yarn forming the conductor may be a graphene yarn. That is, a yarn constructed entirely, essentially or substantially of graphene.

The power source may be a battery. The battery may be a rechargeable battery. The battery may be a rechargeable battery adapted to be charged wirelessly such as by inductive charging.

The power source may comprise an energy harvesting device. The energy harvesting device may be configured to generate electric power signals in response to kinetic events such as kinetic events performed by a wearer of a garment that the biosensing textile forms or is incorporated into. The kinetic event could include walking, running, exercising or respiration of the wearer. The energy harvesting material may comprise a piezoelectric material which generates electricity in response to mechanical deformation of the converter. The energy harvesting device may harvest energy from body heat of a wearer of a garment that the biosensing textile forms or is incorporated into. The energy harvesting device may be a thermoelectric energy harvesting device.

The garment or the inner biosensing textile may further comprise a communicator arranged to communicate with an external device over a wireless network. The communicator may be arranged to communicative over a cellular network. That is, the wireless network may be a cellular network. The cellular network may be a fourth generation (4G) or fifth generation (5G) cellular network or sixth generation (6) cellular network or any other future cellular communication network. The communicator may be incorporated into the controller. The communicator may be provided on the textile panel such as at a location different to that of the controller.

The garment and/or textile panel may be constructed from a woven or a non-woven material. The textile panel may be a fabric panel. The garment and/or textile panel may be formed from yarn. The yarn may be a natural fibre or a natural fibre blended with one or more other materials which can be natural or synthetic or a synthetic fibre. The yarn may be cotton. The cotton may be blended with polyester and/or viscose and/or polyamide according to the particular application. Silk may also be used as the natural fibre. Cellulose, wool, hemp and jute are also natural fibres that may be used in the textile. Polyester, polycotton, nylon and viscose are synthetic fibres that may be used in the textile. The garment and/or textile panel may comprise a synthetic 2-way stretch woven fabric panel.

The biosensing unit may be use for measuring one or a combination of bioelectrical, bioimpedance, biochemical, biomechanical, bioacoustics, biooptical or biothermal signals of the wearer. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer's sweat. The biomechanical measurements include blood pressure. The bioacoustics measurements include phonocardiograms (PCG). The biooptical measurements include orthopantomogram (OPG). The biothermal measurements include skin temperature and core body temperature measurements.

Here, the garment may refer to an item of clothing or apparel. The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, swimwear, wetsuit or drysuit.

The textile panel may comprise a mesh or webbing material. The textile panel and in particular the mesh or webbing material may comprise a raw edge. The textile panel may comprise a first section formed of a raw edge material and a second edge formed of a woven material.

According to a second aspect of the disclosure, there is provided a method of manufacturing a garment. The method comprises providing a garment. The method comprises providing an inner biosensing textile. The inner biosensing textile comprises: a textile panel; and biosensing unit positioned on the textile panel. The method further comprises disposing the inner biosensing textile within the garment. The method further comprises attaching a first region of the textile panel to the garment such that the first region is unable to move relative to the garment. A second region of the textile panel is able to move relative to garment.

According to a third aspect of the disclosure, there is provided a biosensing garment. The biosensing garment comprises a garment. The biosensing garment comprises an inner biosensing textile disposed within the garment. The inner biosensing textile comprises a textile panel. The inner biosensing textile comprises a biosensing unit positioned on the textile panel. The biosensing textile comprises means to position the biosensing unit away from the garment such that, when worn, the biosensing unit is positioned on or near the body surface. The biosensing garment may comprise additional features as disclosed above in relation to the first aspect of the disclosure.

According to a fourth aspect of the disclosure, there is provided a method of manufacturing a garment. The method comprises providing a garment. The method comprises providing an inner biosensing textile. The inner biosensing textile comprises a textile panel. The inner biosensing textile comprises a biosensing unit positioned on the textile panel. The biosensing textile comprises means to position the biosensing unit away from the garment such that, when worn, the biosensing unit is positioned on or near the body surface. The method comprises disposing the inner biosensing textile within the garment. The method comprises attaching the inner biosensing textile to the garment.

According to a fifth aspect of the disclosure, there is provided a biosensing textile arranged to be disposed within a garment. The biosensing textile comprises a textile panel. The biosensing textile comprises a biosensing unit positioned on the textile panel. The textile panel comprises means to position the biosensing unit away from a planar surface of the textile panel. In this way, the textile panel urged towards the body surface when worn. The textile panel may comprise additional features as disclosed above in relation to the first aspect of the disclosure.

According to a sixth aspect of the disclosure, there is provided a method of manufacturing a biosensing textile. The method comprises providing a textile panel. A biosensing unit is positioned on the textile panel. The method comprises providing means to position the biosensing unit away from a planar surface of the textile panel.

According to a seventh aspect of the disclosure, there is provided a biosensing garment. The biosensing garment comprises a garment. The biosensing garment comprises an inner biosensing textile disposed within the garment. The inner biosensing textile comprises a textile panel. The inner biosensing textile comprises an electronic component positioned on the textile panel. The garment further comprises an access region at a location corresponding to the location of the electronic component on the textile panel, wherein the access region is arranged such that at least a part of the electronic component is visible on an outside surface of the garment.

The inner biosensing textile may comprise an electrode positioned on the textile panel.

The access region may be an aperture, transparent region or translucent region on the garment.

The electronic component may comprise a power source. The electronic component may comprise a controller for controlling a biosensing unit. The electronic component may comprise both the power source and the controller. The electronic component may comprise a display. The display may be arranged to indicate a status of the biosensing garment. The display may be arranged to display information relating to the data recorded by the biosensing garment.

The electronic component may comprise an indicator arranged to indicate a status of the electronic component.

The garment may comprise the aperture, and the aperture may be sized to receive the electronic component such that at least a part of the electronic component is accessible via the outside surface of the garment. The electronic component may be removable from the textile panel. The textile panel may comprise a holder for receiving the power source.

The textile panel may comprise a holder arranged to releasably hold the electronic component. The electronic component may be incorporated into or have the appearance of a fastener. The fastener may be a button, clasp, toggle, stud, snap fastener, popper, eyelet or, buckle.

The biosensing garment may comprise additional features as disclosed above in relation to the first aspect of the disclosure.

According to an eighth aspect of the disclosure, there is provided a method of manufacturing a garment. The method comprises providing a garment. The method comprises providing an inner biosensing textile. The inner biosensing textile comprises a textile panel. The inner biosensing textile comprises an electronic component positioned on the textile panel. The garment further comprises an access region at a location corresponding to the location of the electronic component on the textile panel. The access region is arranged such that at least a part of the electronic component is visible on an outside surface of the garment. The method comprises disposing the inner biosensing textile within the garment. The method comprises attaching the inner biosensing textile to the garment.

According to a ninth aspect of the disclosure, there is provided a textile panel suitable for use in the biosensing garment of the first, third, or seventh aspect of the disclosure.

According to a tenth aspect of the disclosure, there is provided a biosensing textile. The biosensing textile comprises a first textile layer and a second textile layer. The second textile layer is attached to a first surface of the first textile layer to define an internal cavity. An electronic component is disposed within the cavity. A biosensing unit is positioned on a second surface of the first textile layer opposite to the first surface of the first textile layer such that the biosensing unit is positioned outside of the internal cavity. The biosensing unit is conductively connected to the electronic component through the first textile layer.

Beneficially, the first and second textile layers define an internal cavity in which the electronic component is disposed. The internal cavity protects the electronic component. The biosensing unit is not disposed within the internal cavity but is instead positioned or incorporated into the first surface of the first textile layer. This enables the biosensing unit to be brought towards or into contact with the skin of a wearer of the biosensing textile so as to enable the biosignal to be measured.

The biosensing textile may form a garment. The biosensing textile may be a biosensing textile as described above in relation to any of the other aspects of the present disclosure.

According to an eleventh aspect of the disclosure, there is provided a method of manufacturing a biosensing textile. The method comprises providing a first textile layer and a second textile layer. The method comprises attaching the second textile layer to a first surface of the first textile layer to define an internal cavity. An electronic component is disposed within the cavity. A biosensing unit for measuring a biosignal is disposed on or integrated into a second surface of the first textile layer opposite to the first surface of the first textile layer such that the biosensing unit is positioned outside of the internal cavity. The biosensing unit is conductively connected to the electronic component through the first textile layer.

According to a twelfth aspect of the disclosure, there is provided a biosensing garment. The biosensing garment comprises a garment in the form of a top. The biosensing garment comprises an inner biosensing textile disposed within the garment. The inner biosensing textile comprises a textile panel. The inner biosensing textile comprises a biosensing unit positioned on the textile panel for measuring a biosignal of the wearer, wherein a pair of first regions of the textile panel are attached to the shoulder regions of the garment such that the first regions are unable to move relative to the garment, and wherein a second region of the textile panel is able to move relative to the garment. The biosensing garment may comprise other features as described in relation to any other aspect of the present disclosure.

### BRIEF DESCRIPTION OF THE DRAWINGS

Examples of the present disclosure will now be described with reference to the accompanying drawings, in which:

FIG. 1 shows a front view of an example garment according to aspects of the present disclosure;

FIG. 2 shows a sectional view of the garment shown in FIG. 1;

FIG. 3 shows a schematic view of an example biosensing textile provided in the garment shown in FIG. 1;

FIG. 4 shows a front sectional view of another example garment according to aspects of the present disclosure;

FIG. 5 shows a rear sectional view of the garment shown in FIG. 4;

FIG. 6 shows a front sectional view of another example garment according to aspects of the present disclosure;

FIG. 7 shows a rear sectional view of the garment shown in FIG. 6;

FIG. 8 shows a side sectional view of the garment shown in FIG. 6;

FIG. 9 shows a front sectional view of another example garment according to aspects of the present disclosure;

FIG. 10 shows a side sectional view of the garment shown in FIG. 9;

FIG. 11 shows a front view of an example garment according to aspects of the present disclosure;

FIG. 12 shows a view of an underside surface of an example biosensing textile according to aspects of the present disclosure;

FIG. 13 shows an internal surface of a pocket of the biosensing textile of FIG. 12

FIG. 14 shows an external view of a pocket of the biosensing textile of FIG. 12;

FIG. 15 shows a flow diagram of an example method of manufacturing a biosensing garment according to aspects of the present disclosure; and

FIG. 16 shows a flow diagram of an example method of manufacturing a biosensing textile according to aspects of the present disclosure;

FIG. 17 shows a flow diagram of another example method of manufacturing a biosensing textile according to aspects of the present disclosure;

FIG. 18 shows a front view of an example garment according to aspects of the present disclosure;

FIG. 19 shows a rear view of the garment shown in FIG. 18.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

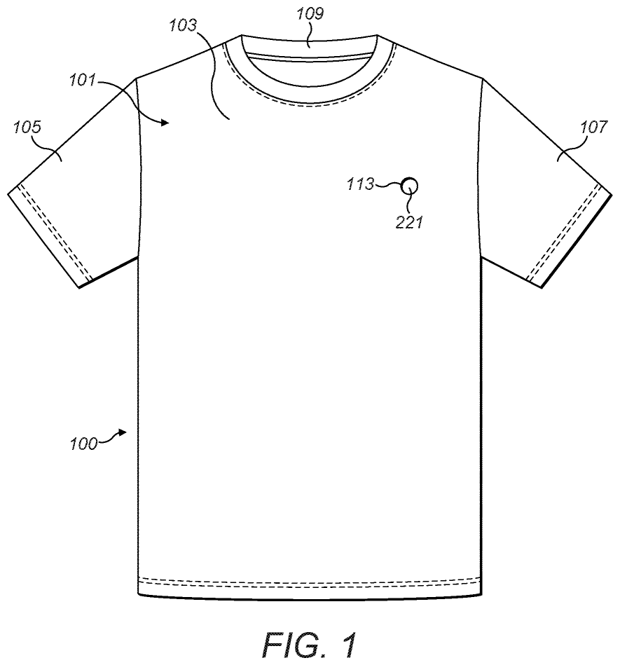

Referring to FIG. 1, there is shown an example biosensing garment 100 according to an aspect of the present disclosure. The biosensing garment 100 comprises a garment 101 in the form of a T-shirt 101. The T-shirt 101 comprises a main body 103, a left sleeve 105, a right sleeve 107 and a collar 109. The T-shirt 100 is a free-form garment. By this it is meant that the T-shirt 100 is loose, not skin-tight, and not a compression garment.

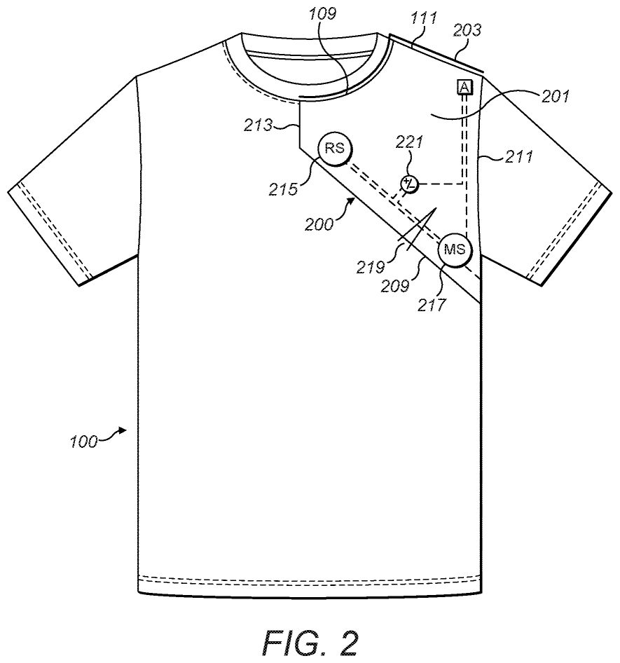

Referring to FIG. 2, there is shown a sectional view of the biosensing garment 100 according to FIG. 1. The sectional view shows that the biosensing garment 100 comprises an inner biosensing textile 200 disposed within the garment 101. The inner biosensing textile 200 is not visible from the outside of the garment 101 and thus does not or does not significantly affect the external appearance of the garment 101.

The biosensing textile 200 comprises a textile panel 201. A first end region 203 of the panel 201 is attached to the garment 101 while the remaining portions of the panel 201 are not attached to the garment 101. This means that while the first end region 203 of the panel 201 is not able to move relative to the garment 101, the remaining regions of the panel 201 are able to move relative to the garment 101. The biosensing textile 200 is able to move freely relative to the garment 101. The panel 201 does not pull on the garment 101 when the wearer moves. This means that the panel 201 does not limit the wearer's mobility and does not affect the outward appearance of the garment 100.

The garment 100 comprises an aperture 113 (FIG. 1) through which a power source 221 of the inner biosensing textile 200 (FIG. 2) is visible. The aperture 113 is sized to receive the power source 221 such that the power source is accessible via the outside surface of the garment 100. The power source 221 may be removable from the textile panel. 291 The textile panel 201 (FIG. 2) may comprise a holder for receiving the power source 221. The power source 221 may snap in/out of the holder or may clip in/out of the holder. The power source 221 may visually indicate the status of the power source 221 such as by indicating the amount of charge remaining for the power source 221. The power source 221 may comprise one or more light sources for indicating the status of the power source 221.

In the example of FIG. 2, the first end region 203 is the top end region 203 of the panel 201 that is attached to the shoulder region 111 and part of the collar region 109 of the garment 101. The remaining portions of the panel 201 are not connected to the garment 101. In this example, the bottom end region 209 and the side regions 211, 213 are free ends, i.e. they are not attached to the garment 101. Beneficially, this means that the panel 201 is attached to the garment 101 at positions corresponding to the shoulder region of the wearer. The shoulder region of the wearer is generally subject to little to no motion even during strenuous exercise. As such, the attachment of the panel 201 to the garment 101 causes little or no pull on the garment 101 even during motion of the wearer.

The panel 201 comprises a plurality of biosensing units 215, 217 in the form of electrodes 215, 217 that are incorporated onto the panel 201. All of the below examples refer to biosensing units 215, 217 in the form of electrodes 215, 217 but the present invention is not limited to this example arrangement. Other forms of biosensing unit 215, 217 are within the scope of the present invention.

The plurality of electrodes 215, 217 are located on an inner surface of the textile panel 201 away from the garment 101 and facing the body surface, when worn. A first of the electrodes 215 is located at a central upper chest region of the panel 201. The “central upper chest region” will be understood as referring to a region which, when worn, corresponds to a central upper chest region of the wearer. Beneficially, when provided in this position, the weight of the electrode 215 causes the panel 201 to hang downwards and urge the first of the electrodes 215 towards the body surface. In this way, the attachment of the panel 201 to the garment 101 causes the electrode 215 to be positioned towards or near the body surface so that the electrode 215 may measure biosignals of the wearer.

A second of the electrodes 217 is located at a lower left chest region of the panel 201. The “lower left chest region” will be understood as referring to a region which, when worn, corresponds to a lower left chest region of the wearer which is proximate to a cardiac region of the wearer. The panel 201 is shaped to position the second of the electrodes 217 away from the garment 101. In this way, when worn, the second electrode 217 is positioned on or near the body surface. The shaping of the panel 201 is achieved through use of a dart 219 in the panel 201. The dart 219 will be understood as referring to a fold that is sewn or otherwise introduced into the panel 201 to provide the shape to the panel 201. The panel 201 may be thought of as having a flat, planar, surface. The dart 219 has the effect of removing a wedge-shaped piece of the panel 201 and pulling the edges of that wedge together to create a shallow cone. In this way, the dart 219 urges the electrode 217 away from the main planar surface of the panel 201.

The dart 219 is not required in all examples of the present disclosure, and instead other structures or features of the panel 201 may be used to provide the desired shape to the panel 201 to position the second of the electrodes 217 away from the garment 101. For example, a seam, pleat, or gather in the panel 201 may be used to provide the same effect as the dart 219.

The panel 201 may be bias cut. This means that that a piece of textile forming the panel 201 is cut diagonally or obliquely to the grain of the textile. Being cut on the bias means that the panel 201 has more give when compared to textiles cut along the straight grain or cross grain so as to accommodate movement. Being bias cut means that the panel 201 will drape in a way which contours to the shape of the body surface. This helps maintain the electrodes 215, 217 in a position which is near or in contact with the body surface.

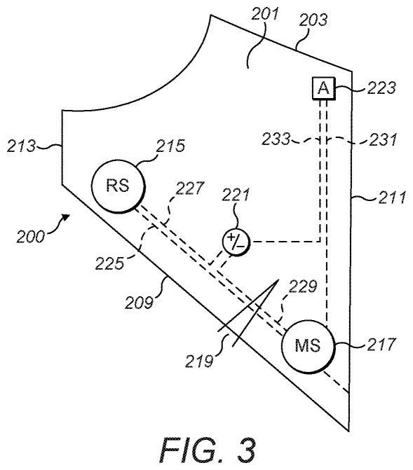

Referring to FIG. 3, there is shown a detailed view of the biosensing textile 200 shown in FIG. 2. The biosensing textile 200 comprises a first electrode 215 and second electrode 217. The electrodes 215, 217 are both included in separate sensor housings which additionally include controllers for controlling the electrodes 215, 217. That is, a first controller for controlling the first electrode 215 and a second controller for controlling the second electrode 217 are provided. The textile panel 201 is shaped to urge the electrode 217 away from a major surface of the textile panel 201 and in particular comprises a dart 219. In this way, the textile panel 201 is shaped to position the electrode 217 toward the body surface when worn.

The first electrode 215 may act as a reference electrode 215. The first controller may act as a reference controller. The second electrode 217 may act as a measuring electrode 217. The second controller may act as a measuring controller. That is, one of the first and second electrodes 215, 217 may be controlled to act as a reference during biopotential and/or bioimpedance measurements. The first electrode 101 and second electrode 105 may be conventional metallic electrodes such as silver/silver chloride (Ag/AgCl) electrodes.

The first controller and the second controller are able to stimulate the body, such as by injecting a current into the body via the electrode(s) 215, 217 for performing an impedance measurement. The first controller and the second controller are also able to measure a physiological signal of the body, such as an ECG, by measuring a potential via the electrode(s) 215, 217. The first electrode 215 and the second electrode 217 may both comprise a first electrical contact and a second electrical contact which are spaced apart from another. The first and second electrical contacts may be arranged as concentric rings, for example. The potential may be measured between the electrical contacts of the first electrode 215 and/or the second electrode 217.

The biosensing textile 200 further comprises a communicator 223. The communicator 223 transmits biodata recorded by the electrodes 215, 217 and optionally processed by the first/second controller wirelessly to an external device. In some examples of the present disclosure, the communicator 223 is a cellular communicator 223 operable to communicate the biometric data wirelessly with an external server via one or more base stations.

The communicator 223 is conductively connected to the second controller by a conductor 231. The communicator 223 in this example is shown at a position which is spaced apart from the first and second electrodes 215, 217 at a position close to the end region 203 of the textile panel 201. In some examples, the communicator 223 may be incorporated with one of the controllers or the electrodes 215, 217. The first electrode 215 and/or first controller are conductively connected to the second electrode 217 and/or second controller via a conductor 225.

The textile panel 201 further comprises a power source 221 for powering the first controller and the second controller. The power source 221 may be a battery 221. The power source 221 is conductively connected to the first controller by a conductor 227. The power source 221 is conductively connected to the second controller by a conductor 229. The power source 221 is conductively connected to the communicator 223 by a conductor 233. In other examples, a separate power source is provided for each of the controllers. That is, a first power source may be provided for powering the first controller and a second power source may be provided for powering the second controller.

The conductors 225, 227, 229, 231, 233 are, in this example, formed of a graphene or a graphene-derivative and are printed onto the textile 200 using a screen-printing process. Other printing processes may be used. In some examples, the conductor 225, 227, 229, 231, 233 may be a conductive transfer. The conductive transfer may comprise graphene.

It will be appreciated that the present disclosure is not limited to screen printing conductors onto a textile or the use of conductive transfers. In other examples, the conductors may be incorporated into one or more fibres of the textile.

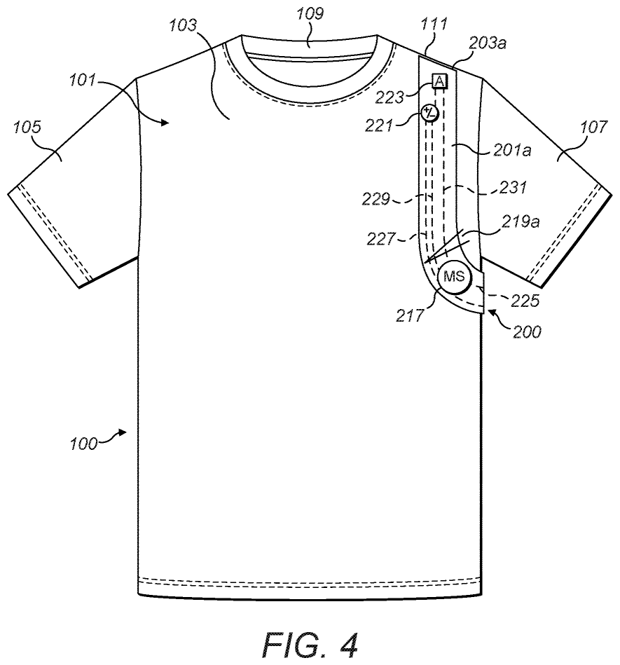

Referring to FIG. 4, there is shown a front sectional view of another example biosensing garment 100 according to aspects of the present disclosure. The sectional view shows that the biosensing garment 100 comprises garment 101 and an inner biosensing textile 200 disposed within the garment 101. The inner biosensing textile 200 is not visible from the outside of the garment 101 and thus does not or does not significantly affect the external appearance of the garment 101.

The inner biosensing textile 200 comprises a first panel 201a. A first end region 203 of the first panel 201a is attached to the garment 101 while the remaining portions of the first panel 201a are not attached to the garment 101. This means that while the first end region 203a of the panel 201a is not able to move relative to the garment 101, the remaining regions of the panel 201a are able to move relative to the garment 101

In the example of FIG. 4, the first end region 203a is the top end region 203a of the panel 201a that is attached to the shoulder region 111 of the garment 101. The remaining portions of the first panel 201a are not connected to the garment 101 and are able to move freely relative to the garment 101.

The end region 203 of the inner biosensing textile 200 is attached to the shoulder region of the garment 101 using a twin needle top stitch that is provided either side of the seam of the garment. The shoulder of the garment 101 which is not attached to the inner biosensing textile 200 is provided with the same or similar twin needle top stitch to even out the visual appearance of the garment.

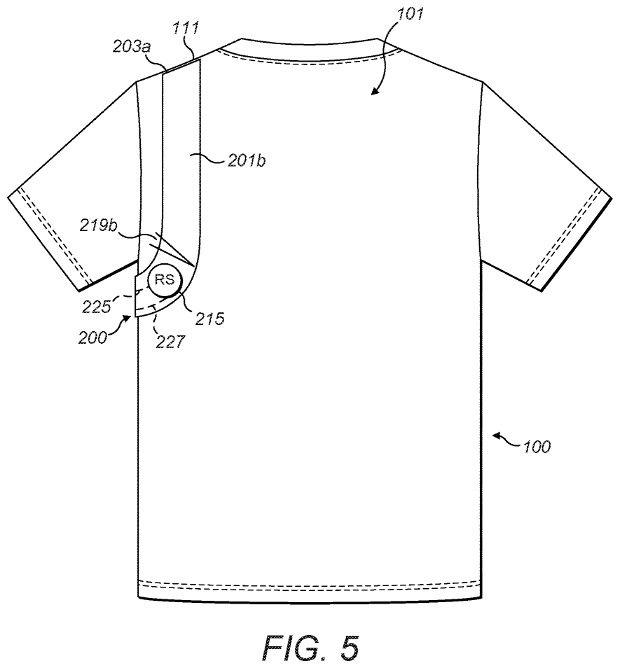

Referring to FIG. 5, there is shown a rear sectional view of the biosensing garment 100 of FIG. 4. The sectional view shows that the inner biosensing textile 200 further comprises a second panel 201b. A first end region 203b of the second panel 201b is attached to the garment 101 at the shoulder region 111. The opposite end region of the second panel 201b is attached to the first panel 201a. In this way, the first panel 201a and the second panel 201b are joined together to define a loop or U-shaped band within the garment 101 that defines an aperture for receiving an upper appendage (e.g. an arm) of the wearer.

The first and second panels 201a, 201b may be attached to another by stiches, staples or adhesive for example. In some examples, the first and second panels 201a, 201b are integrally formed from a single unitary piece of textile.

The biosensing textile 200 comprises a plurality of electrodes 215, 217 that are incorporated onto the panels 201a, 201b. A first of the electrodes 215 is located at a lower left chest region of the second panel 201b (FIG. 5). The second panel 201b is shaped to position the first of the electrodes 215 away from the garment 101 and towards the body surface. In this way, when worn, the first electrode 215 is positioned on or near the body surface. The shaping of the second panel 201b is achieved through use of a dart 219b in the second panel 201b. A second of the electrodes 217 is located at the lower left chest region of the first panel 201a (FIG. 4). The first panel 201a is shaped to position the second electrode 217 away from the garment 101 and towards the body surface. In this way, when worn, the second electrode 217 is positioned on or near the body surface. The shaping of the first panel 201a is achieved through use of a dart 219a in the first panel 201a.

The first electrode 215 is provided with a first controller in a sensor housing. The second electrode 217 is provided with a second controller in a sensor housing.

The biosensing textile 200 further comprises a communicator 223. The communicator 223 is provided on the first panel 201a in this example. The communicator 223 is conductively connected to the second controller by a conductor 231.

The communicator 223 in this example is shown at a position which is spaced apart from the first and second electrodes 215, 217 at a position close to the end region 203a of the first panel 201a. The first electrode 215 and/or first controller are conductively connected to the second electrode 217 and/or second controller via a conductor 225 that extends between the first panel 201a and the second panel 201b.

The first panel 201a further comprises a power source 221 for powering the first controller and the second controller. The power source 221 is conductively connected to the first controller by a conductor 227. The power source 221 is conductively connected to the second controller by a conductor 229. The power source 221 is conductively connected to the communicator 223 by a conductor 233.

In other examples, a separate power source is provided for each of the controllers. That is, a first power source may be provided for powering the first controller and a second power source may be provided for powering the second controller.

The conductors 225, 227, 229, 231, 233 are also formed of a graphene or a graphene-derivative and are printed onto the textile 200 using a screen-printing process. Other printing processes may be used. In some examples, the conductor 225, 227, 229, 231, 233 may be a conductive transfer. The conductive transfer may comprise graphene.

The first panel 201a and the second panel 201b may be bias cut. This means that that a piece of textile forming the panel 201a, 201b is cut diagonally or obliquely to the grain of the textile. Being cut on the bias means that the panel 201a, 201b has more give when compared to textiles cut along the straight grain or cross grain so as to accommodate movement. Being bias cut means that the panel 201a, 201b will drape in a way which contours to the shape of the body surface. This helps maintain the electrodes 215, 217 in a position which is near or in contact with the body surface.

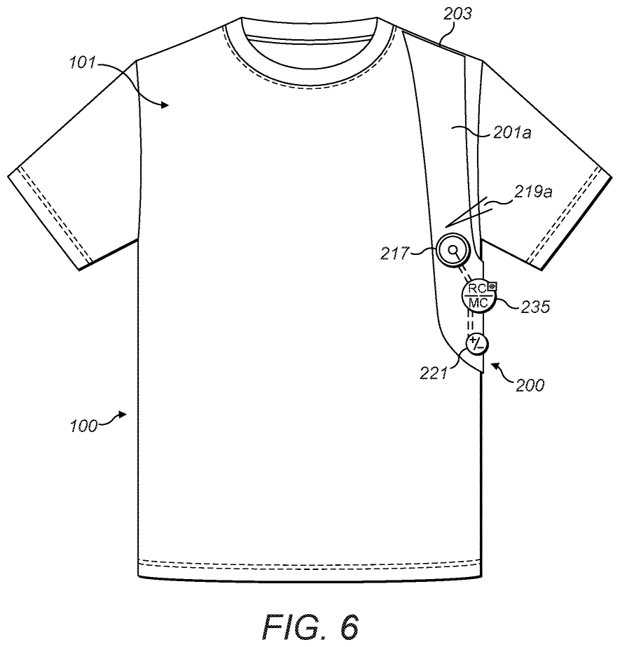

Referring to FIG. 6, there is shown a front sectional view of another example biosensing garment 100 according to aspects of the present disclosure. The sectional view shows that the biosensing garment 100 comprises an inner biosensing textile 200 disposed within the garment 101. The inner biosensing textile 200 is not visible from the outside of the garment 101 and thus does not or does not significantly affect the external appearance of the garment 101.

The inner biosensing textile 200 comprises a first panel 201a. A first end region 203 of the first panel 201a is attached to the garment 101 while the remaining portions of the first panel 201 are not attached to the garment 101. This means that while the first end region 203a of the panel 201a is not able to move relative to the garment 101, the remaining regions of the garment panel 201a are able to move relative to the garment 101. In the example of FIG. 6, the first end region 203a is the top end region 203a of the panel 201a that is attached to the shoulder region 111 of the garment 101. The remaining portions of the first panel 201a are not connected to the garment 101.

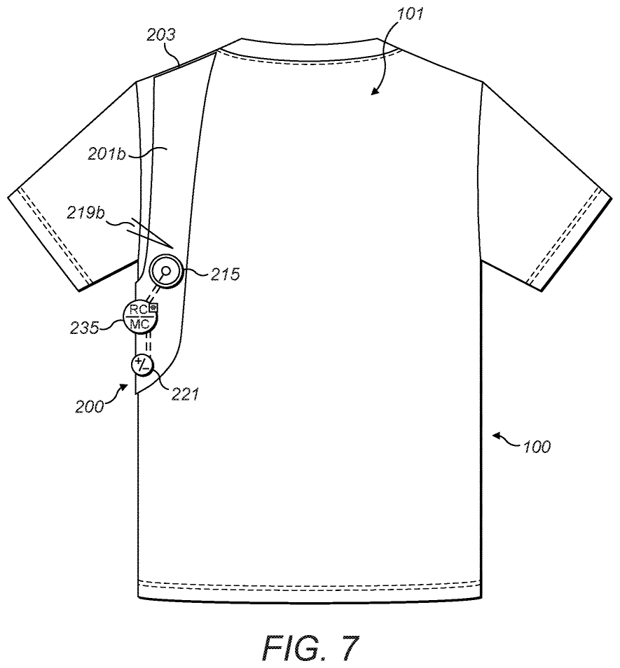

Referring to FIG. 7, there is shown a rear sectional view of the biosensing garment 100 of FIG. 6. The sectional view shows that the inner biosensing textile 200 further comprises a second panel 201b. A first end region 203b of the second panel 201b is attached to the garment 101 at the shoulder region 111. The opposite end region of the second panel 201b is attached to the first panel 201a and in this example is integrally formed with the first panel 201a. That is the first panel 201a and the second panel 201b form a single, integral structure. In this way, the first panel 201a and the second panel 201b are joined together to define a loop U-shaped band within the garment 101 that defines an aperture for receiving an upper appendage (e.g. an arm) of the wearer.

The biosensing textile 200 comprises a plurality of electrodes 215, 217 that are incorporated onto the panels 201a, 201b. A first of the electrodes 215 is located at a lower left chest region of the second panel 201b (FIG. 7). The second panel 201b is shaped to position the first of the electrodes 215 away from the garment 101 and towards the body surface. In this way, when worn, the first electrode 215 is positioned on or near the body surface. The shaping of the second panel 201b is achieved through use of a dart 219b in the second panel 201b. A second of the electrodes 217 is located at the lower left chest region of the first panel 201a (FIG. 7). The first panel 201a is shaped to position the second electrode 217 away from the garment 101 and towards the body surface. In this way, when worn, the second electrode 217 is positioned on or near the body surface. The shaping of the first panel 201a is achieved through use of a dart 219a in the first panel 201a.

In this example, a single controller 235 is provided for controlling the first electrode 215 and the second electrode 217. The single controller 235 is separated from the first electrode 215 and the second electrode 217.

Importantly, in the example shown in FIGS. 6 and 7, the first electrode 215 is located at a first position on the textile 200. The second electrode 217 is located at a second position on the textile 200. The first position is spaced apart from the second position. Moreover, the controller 235 is located at a third position on the textile 200. The third position is spaced apart from the first position, second position, and third position. In this way, the electrodes 215, 217 are spaced apart from one another and from the controller 235. Beneficially, distributing the components on the textile 200 such as by separating the electrodes 215, 217 and controller 235 and separating the electrodes 215, 217 from the controller 235 can reduce the apparent footprint of the components on the textile 200. Existing solutions provide the electrodes, controllers, and power sources as integrated units within a housing that this then attached to the textile. This integrated units are bulky and protrude outward from the textile. This may make the resultant garments uncomfortable to wear and unattractive.

The biosensing textile 200 further comprises a communicator 223. The communicator 223 is provided within the controller 235 in this example. The biosensing textile 200 further comprises a power source 221 for powering the controller 235. The power source 221 is conductively connected to the controller 235.

The conductors connecting the controller 235 to the electrodes 215, 217, and the controller 235 to the power source 221 may be formed of a graphene or a graphene-derivative and are printed onto the textile 200 using a screen-printing process. Other printing processes may be used. In some examples, the conductors may be formed of a conductive transfer. The conductive transfer may comprise graphene.

The first panel 201a and the second panel 201b may be bias cut. This means that that a piece of textile forming the panel 201a, 201b is cut diagonally or obliquely to the grain of the textile. Being cut on the bias means that the panel 201a, 201b has more give when compared to textiles cut along the straight grain or cross grain so as to accommodate movement. Being bias cut means that the panel 201a, 201b will drape in a way which contours to the shape of the body surface. This helps maintain the electrodes 215, 217 in a position which is near or in contact with the body surface.

The end region 203 of the inner biosensing textile 200 is attached to the shoulder region of the garment 101 using a twin needle top stitch that is provided either side of the seam of the garment. The shoulder of the garment 101 which is not attached to the inner biosensing textile (200) is provided with the same or similar twin needle top stitch to even out the visual appearance of the garment.

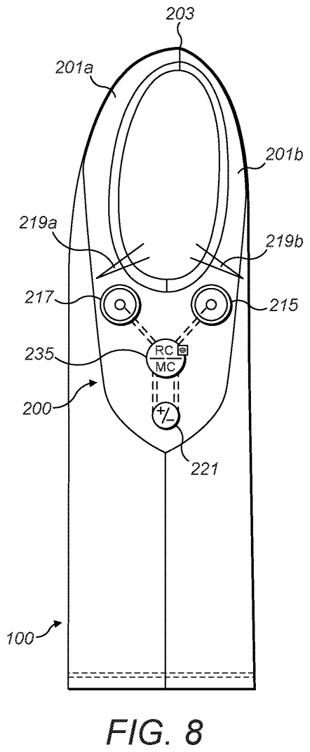

Referring to FIG. 8, there is shown a side view of the biosensing garment 100 shown in FIGS. 6 and 7. Here, it can be seen that the first panel 201a and the second panel 201b are formed from a single unitary piece of textile material.

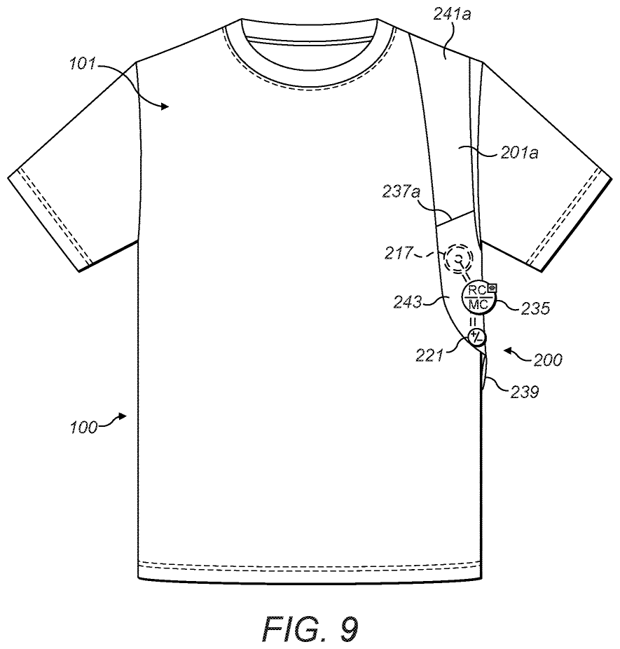

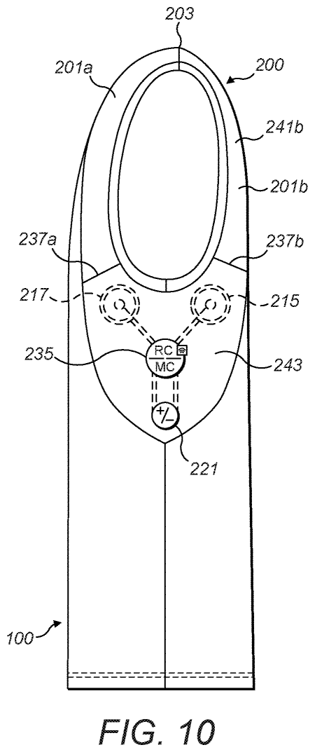

Referring to FIGS. 9 and 10, there is shown a front sectional view and side sectional view of another example biosensing garment 100 according to aspects of the present disclosure The first electrode 215 and the second electrode 217 are incorporated onto the textile 200. Being “incorporated” onto the textile 200 may mean that the electrodes 215, 217 are printed onto the textile 200 or are formed from one or more fibres or yarns of the textile 200 such as by coating the fibres or yarns of the textile 200 with an electrically conductive material. Importantly still, the first and second electrodes 215, 217 in the example of FIGS. 9 and 10 are formed of a 2D electrically conductive material. In the example of FIGS. 9 and 10, this material is a graphene or graphene-derivative which is screen printed onto the textile 200. The combination of the electrodes 215, 217 being integrated into the textile 200 and formed of a 2D electrically conductive material means that the electrodes 215, 217 have a minimal footprint on the textile 200. This means that the impact of the sensing system 100 on the textile 200 is minimised. The textile 200 is more comfortable when worn as there are no or only minimal bulky electronics components.

The textile panels 201a, 201b comprise seams 237a, 237b for positioning the electrodes 215, 217 towards the body surface.

The inner biosensing textile 200 further comprises a fastener 239 in the form of a loose tacking loop that loosely attaches the biosensing textile 200 to the garment 101. The fastener 239 still allows the biosensing textile 200 to move freely relative to the garment 101.

The textile panel 201a comprises a first textile layer 241a. The electrode 217 is provided on the underside surface (second surface) of the first textile layer 241a such that the electrode 217 is proximate to the skin of the wearer. The textile panel 201b comprises a first textile layer 241b. The electrode 215 is provided on the underside surface (second surface) of the first textile layer 241b such that the electrode 215 is proximate to the skin of the wearer. The ends 203 of the first textile layers 241a, 241b are attached to the shoulder region of the garment 101. The other ends of the first textile layers 241a, 241b are attached together. The controller 235 and power source 221 are provided on the first surface of the first textile layers 241a, 241b opposite to the second surface of the first textile layers 241a, 241b. Conductive connections extend through the first textile layers 241a, 241b to conductively connect the controller 235 and power source 221 to the electrodes 215, 217. A second textile layer 243 is provided on top of the first surface of the first textile layers 241a, 241b and attached at the edges of the first textile layers 241a, 241b to define an internal cavity (pocket) in which the controller 235 and power source 221 are provided. The edges of the second textile layer 243 running parallel to the seams 237a, 237b are not attached to the first textile layer 241a, 241b. This provides openings via which the controller 235 and power source 221 may be accessed.

In other examples, all of the edges of the second textile layer 243 are joined to the first textile layers 241a, 241b to form an enclosed space in which the electronic components are provided. This helps protect against water ingress especially if the first and second textile layers are constructed from a waterproof material.

In an example method of manufacture, the electrodes 215, 217 are attached to the second surface of the first textile layers 241a, 241b. The electronic component is positioned on the first surface of the first textile layers 241a, 241b and the electrical connection connecting the electrodes 215, 217 to the controller 235 are formed. The first textile layer 241a, 241b is then sewn to the second textile layer 243 inside out. The textile layers 241a, 241b, 243 are then inverted to be the right way out. This process is known as bagging out.

The end region 203 of the inner biosensing textile 200 is attached to the shoulder region of the garment 101 using a twin needle top stitch that is provided either side of the seam of the garment. The shoulder of the garment 101 which is not attached to the inner biosensing textile is provided with the same or similar twin needle top stitch to even out the visual appearance of the garment.

The other components of the garment 100 are the same as the garment 100 of FIGS. 6, 7 and 8.

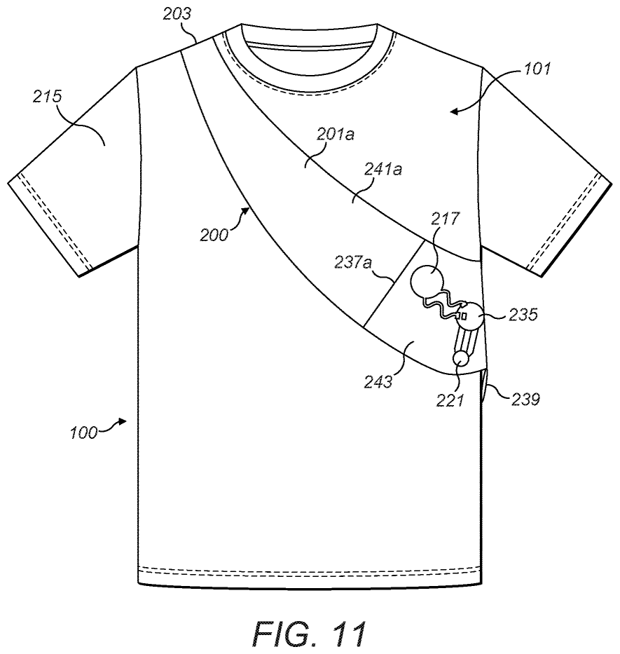

Referring to FIG. 11, there is shown another example biosensing garment 100 according to an aspect of the present disclosure. The biosensing garment 100 comprises a garment 101 in the form of a T-shirt 101. The inner biosensing textile 200 is in the form of a sash that extends across the chest of the wearer. The inner biosensing textile 200 is therefore an over-the-shoulder sash 200.

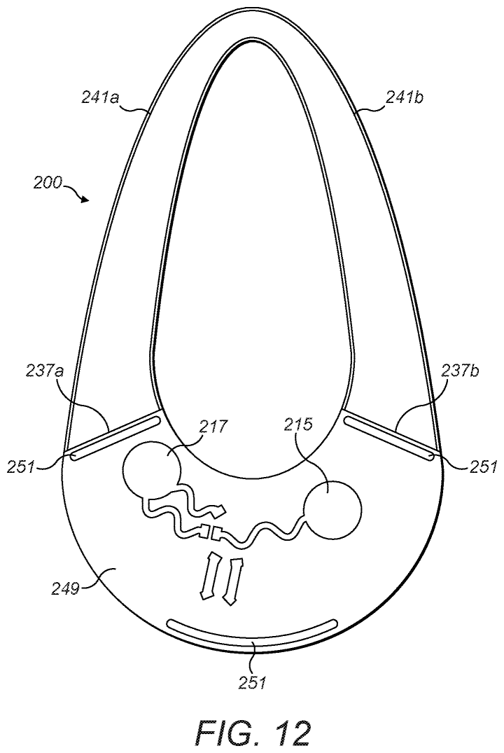

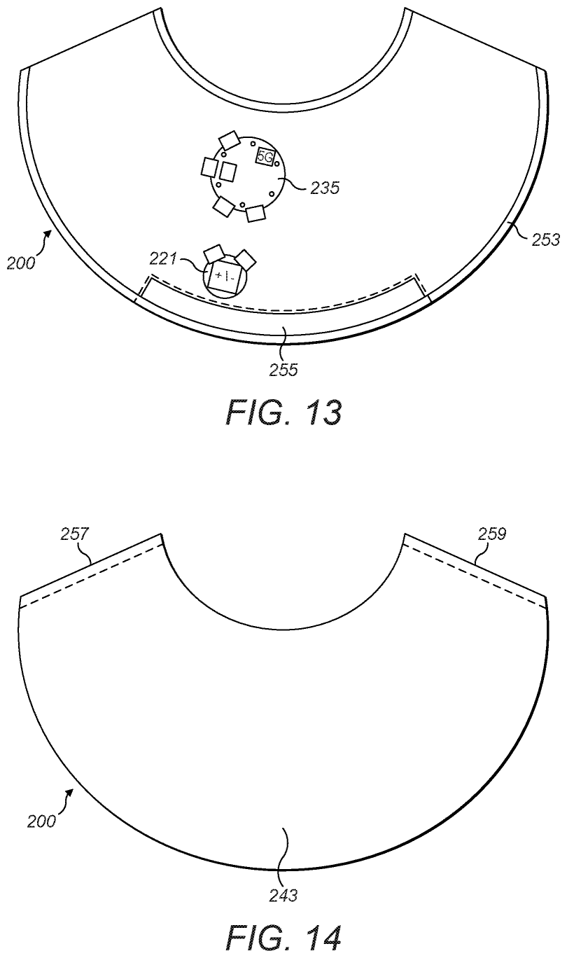

The inner biosensing textile 200 comprises a first panel 201a (FIG. 12) located on the front side of the wearer and a second panel 201b (FIG. 13) located on the rear side of the wearer. The first panel 201a and the second panel 201b are connected to the garment 101 at an end region 203. The remaining portions of the inner biosensing textile 200 are not attached to the garment 101 and are free to move relative to the garment 101. The inner biosensing textile 200 further comprises a fastener 239 in the form of a loose tacking loop that loosely attaches the biosensing textile 200 to the garment 101. The fastener 239 still allows the biosensing textile 200 to move freely relative to the garment 101.

The first panel 201a and the second panel 201b comprise first sections formed of a raw edge mesh material and a second edge formed of a woven material. The raw edge (i.e. no seam) is beneficial in minimizing the effect of the inner biosensing textile 200 on the outward visual appearance of the garment 101. The mesh material is joined to the woven material by seams 237a, 237b.

The sash 200 naturally hangs due to gravity in a way that urges the electrodes 215, 217 towards the body surface. The seams 237a, 237b are provided to further aid in urging the electrodes 215, 217 towards the body surface.

The first panel 201a and the second panel 201b further comprise an internal cavity (pocket) formed by first textile layers 241a, 241b and second textile layer 243 in which the controller 235, and power source 221 are provided.

The end region 203 of the inner biosensing textile 200 is attached to the shoulder region of the garment 101 using a twin needle top stitch that is provided either side of the seam of the garment. The shoulder of the garment 101 which is not attached to the inner biosensing textile 200 is provided with the same or similar twin needle top stitch to even out the visual appearance of the garment.

Referring to FIG. 12, there is shown a side view of the biosensing textile 200 of FIG. 11. The view in FIG. 12 shows the underside surface (second surface) of the biosensing textile 200 that faces then skin of the wearer when worn. In FIG. 12, it can be seen that the electrodes 215, 217 are provided on the underside surface 249 of the pocket that faces the skin surface along with the conductive tracking that connects the electrodes 215, 217 to the controller 235 (FIG. 13) and the power source 221 (FIG. 13) to the controller 235. In other words, the electrodes 215, 217 and the conductive tracking are provided on the internal surface of the first textile layers 241a, 241b that face the skin surface. The underside surface 249 of the pocket further comprises gripper sections 251 in the form of silicone tape 251 that help hold and maintain the electrodes 215, 217 near or in contact with the skin surface of the wearer. The underside surface 249 of the pocket is made from a woven material while the remaining sections of the first textile layers 241a, 241b are made from a mesh material.

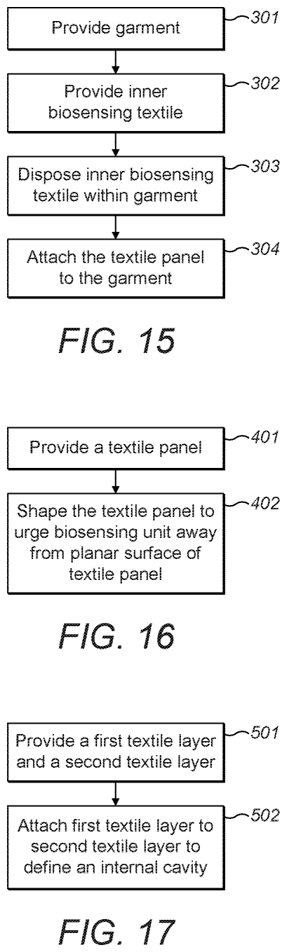

Referring to FIG. 13, there is shown the internal surface (first surface) 253 of the pocket. That is, the surface 253 of the first textile layers 241a, 241b that is covered by the second textile layer 243 (FIG. 15) to form the internal cavity. FIG. 13 shows that the controller 235 and the power source 221 are provided on the internal surface 253 of the pocket and are therefore disposed within the internal cavity of the pocket. Conductive connections extend through the first textile layers 241a, 241b to conductively connect the components provided on either side of the textile layer. The internal surface 253 further comprises a weight 255 that is stitched or otherwise fixed in position in the pocket. The weight 255 helps to urge the electrodes 215, 217 (FIG. 12) towards or in contact with the skin surface of the wearer.

Referring to FIG. 14, there is shown an outer surface of the pocket formed by the second textile layer 243. The second textile layer 243 is attached to the first textile layers 241a, 241b around the edges. In some examples, the second textile layer 243 is not joined to the first textile layers 241a, 241b at the ends 257, 259 so that access openings are provided to allow for the power source 221 and or controller 235 to be accessed.

Referring to FIG. 15, there is shown an example method of manufacturing a biosensing garment according to aspects of the present disclosure. Step 301 of the method comprises providing a garment. Step 302 of the method comprises providing an inner biosensing textile. Step 303 of the method comprises disposing the inner biosensing textile within the garment. Step 304 of the method comprises attaching the textile panel to the garment.

Referring to FIG. 16, there is shown an example method of manufacturing a biosensing textile according to aspects of the present disclosure. Step 401 of the method comprises providing a textile panel. Step 402 of the method comprises shaping the textile panel to urge biosensing unit away from the planar surface of the textile panel.

Referring to FIG. 17, there is shown an example method of manufacturing a biosensing textile according to aspects of the present disclosure. Step 501 of the method comprises providing a first textile layer and a second textile layer. Step 502 of the method comprises attaching the first textile layer to the second textile layer to define an internal cavity.

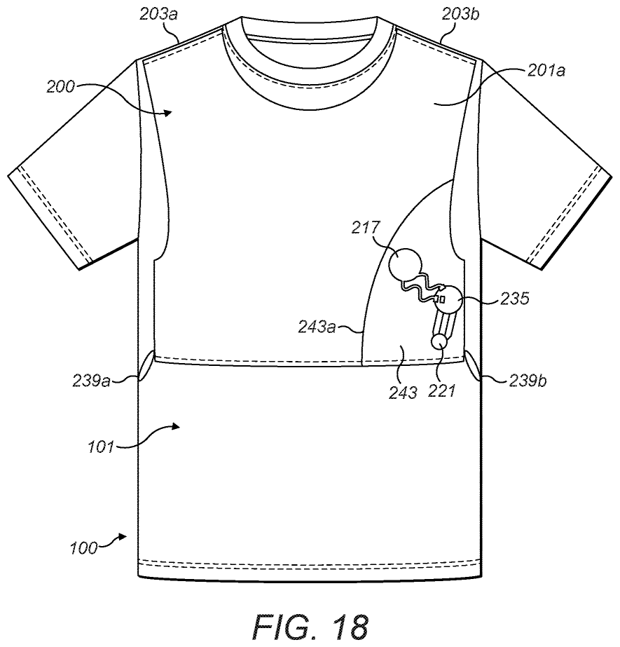

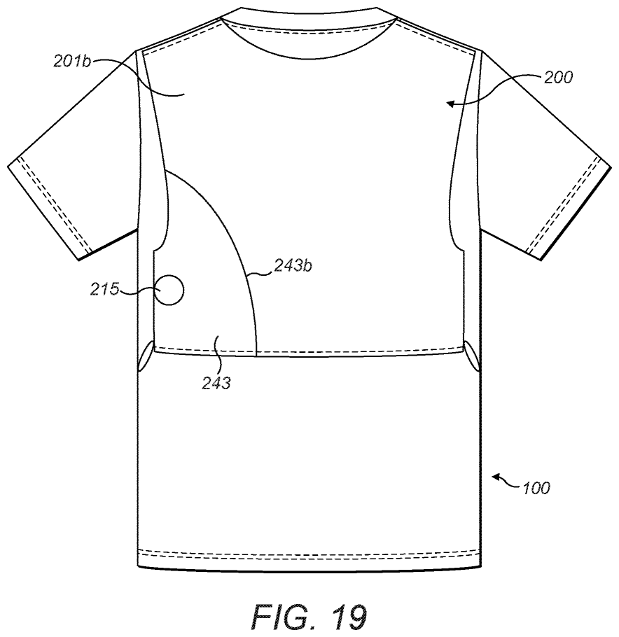

Referring to FIGS. 18 and 19, there is shown a front view (FIG. 18) and a rear view (FIG. 19) of another biosensing garment 100 according to an aspect of the present disclosure. The biosensing garment 100 comprises a garment 101 in the form of a T-shirt 101. The inner biosensing textile 200 is in the form of a crop that covers the front and back upper chest regions of the wearer. The inner biosensing textile 200 is attached to the garment 101 at the two shoulder regions 203a, 203b of the garment using a twin needle top stitch. Two fasteners 239a, 239b are also provided to loosely fasten the lower edge of the biosensing textile 200 to the garment 101. The fasteners 239a, 239b are in the form of loose tacking loops. The fasteners 239a, 239b still allow the biosensing textile 200 to move freely relative to the garment 101.

The biosensing textile 200 comprises a front panel 201a and a back panel 201b. The front panel 201a is arranged to cover the front upper half of the chest when worn. The back panel 201b is arranged to cover the back upper half of the chest when worn. The front panel 201a and the back panel 201b are attached to one another and define armholes through which the arms may pass through when worn.

The biosensing textile 200 comprises a pocket 243 arranged to house electronic components and in particular houses the controller 235 and power source 221. The pocket 243 is formed by a first textile layer and a second textile layer positioned adjacent to and external to the first textile layer. The electrodes 215, 217 are provided on the underside surface of the first textile such that they are arranged to be positioned proximate to or in contact with the skin surface of the wearer when worn.

The front panel 201a and the back panel 201b are formed of a raw edge mesh material. The first and second textile layers formed the pocket 243 are made of a woven textile material. The woven textile material in this example is cut on the grain. The biosensing textile 200 in the form of a crop provides a tighter fit that then outer garment 101 so as to help hold the biosensing unit(s) in close proximity/skin contact with the skin surface of the wearer. Other features of the garment shown in FIGS. 18 and 19 may be the same as the example garments described above.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A biosensing garment comprising:
a garment; and
an inner biosensing textile disposed within the garment, the inner biosensing textile comprising:
a textile panel;
a biosensing unit positioned on the textile panel for measuring a biosignal of the wearer; and
a holder arranged to releasably hold an electronic component,
wherein a first region of the textile panel is attached to the garment such that the first region is unable to move relative to the garment, and wherein a second region of the textile panel is able to move relative to the garment.

2. The biosensing garment as claimed in claim 1, wherein the holder comprises a first textile layer and a second textile layer, wherein the second textile layer is attached to the first textile layer to define an internal cavity for receiving the electronic component.

3. The biosensing garment as claimed in claim 2, wherein the first textile layer comprises a first surface that faces in to the internal cavity and a second surface that faces away from the internal cavity, and wherein the biosensing unit is positioned on the second surface.

4. The biosensing garment as claimed in claim 3, wherein the biosensing unit is conductively connected to the electronic component through the first textile layer.

5. The biosensing garment as claimed in claim 2, wherein the internal cavity is in the form of a pocket with an opening such that at least part of the electronic component may be accessed and/or removed.

6. The biosensing garment as claimed in claim 2, wherein the second textile layer is adhered or welded to the first textile layer.

7. The biosensing garment as claimed in claim 1, wherein the electronic component comprises one or more of a power source, a controller, and a communicator for communicating with an external device.

8. The biosensing garment as claimed in claim 1, wherein the textile panel is shaped to position the biosensing unit away from the garment such that, when worn, the biosensing unit is positioned on or near the body surface.

9. The biosensing garment as claimed in claim 8, wherein the textile panel comprises a dart, wherein the dart acts to shape the textile such that the biosensing unit is positioned away from the garment.

10. The biosensing garment as claimed in claim 8, wherein the textile panel comprises a seam, wherein the seam acts to shape the textile such that the biosensing unit is positioned away from the garment.

11. The biosensing garment as claimed in any of claim 8, wherein the biosensing textile comprises a weight for urging the textile panel down and towards a wearer of the garment.

12. The biosensing garment as claimed in claim 1, wherein the biosensing textile comprises a gripper section, wherein the gripper section is provided on the underside surface of the biosensing textile and is arranged to grip the biosensing textile to a skin surface of a wearer of the garment.

13. The biosensing garment as claimed in claim 1, wherein the textile panel is bias cut.

14. (canceled)

15. A method of manufacturing a garment, comprising:
providing a garment;
providing an inner biosensing textile, the inner biosensing textile comprising: a textile panel; a biosensing unit positioned on the textile panel for measuring a biosignal of a wearer; and a holder arranged to releasably hold an electronic component;
disposing the inner biosensing textile within the garment;
attaching a first region of the textile panel to the garment such that the first region is unable to move relative to the garment, and wherein a second region of the textile panel is able to move relative to the garment.

16. A biosensing garment comprising:
a garment; and
an inner biosensing textile disposed within the garment, the inner biosensing textile comprising:
a textile panel forming a pocket having an internal cavity, the pocket having an opening sized such that an electronic component is able to be inserted into and removed from the internal cavity of the pocket;
a biosensing unit positioned on the textile panel for measuring a biosignal of the wearer; and
a gripper section, wherein the gripper section is provided on the underside surface of the pocket that faces a skin surface of a wearer of the garment and is arranged to grip the biosensing textile to the skin surface.
